(19) United States
(12) Patent Application Publication
(10) Pub. No.: US 2024/0415569 A1
(43) Pub. Date: Dec. 19, 2024

(54) DUAL MATERIAL NEEDLES FOR NEEDLE-BASED NEUROMODULATION

(71) Applicant: Medtronic Ireland Manufacturing Unlimited Company, Dublin (IE)

(72) Inventor: Kelsey Sandquist, Cotati, CA (US)

(21) Appl. No.: 18/701,459

(22) PCT Filed: Oct. 18, 2022
(86) PCT No.: PCT/EP2022/078961

§ 371 Date: Apr. 15, 2024

Related U.S. Application Data
(60) Provisional application No. 63/272,038, filed on Oct. 26, 2021.

(51) Int. Cl. A61B 18/14; A61B 18/00

(52) U.S. Cl. CPC A61B 18/1492; A61B 2018/00077; A61B 2018/00083; A61B 2018/00184; A61B 2018/00434; A61B 2018/143; A61B 2018/1465; A61B 2018/1475; A61B 2218/002

(57) ABSTRACT

A neuromodulation catheter may include an elongated member configured to be navigated through vasculature of a patient to a target treatment site and a plurality of therapeutic elements arranged around a distal portion of the elongated member. Each therapeutic element may include a needle including a first portion comprising a polymer and a second portion including a metal. The second portion is configured to puncture a vessel wall at the target treatment site when the needle is extended radially relative to a longitudinal axis of the elongated member.

### TECHNICAL FIELD

The present technology is related to neuromodulation using therapeutic elements that include needles.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

### SUMMARY

The present technology is directed to devices, systems, and methods for neuromodulation, such as renal neuromodulation.

In some examples, the disclosure describes a neuromodulation catheter, the catheter comprising an elongated member configured to be navigated through vasculature of a patient to a target treatment site and a plurality of therapeutic elements arranged around a distal portion of the elongated member. Each therapeutic element may include a needle including a first portion comprising a polymer and a second portion including a metal. The second portion is configured to puncture a vessel wall at the target treatment site when the needle is extended radially relative to a longitudinal axis of the elongated member.

In some examples, the disclosure describes a method that includes navigating a catheter through vasculature of a patient to a target treatment site. The catheter includes an elongated member and a plurality of therapeutic elements arranged around a distal portion of the elongated member. Each therapeutic element of the plurality of therapeutic element may include a needle including a first portion including a polymer and a second portion including a metal. The method also may include extending at least one of the plurality of needles radially relative to a longitudinal axis of the elongated member and puncturing a vessel wall at the target treatment site of the patient using the second portion.

Also disclosed herein is neuromodulation catheter that includes an elongated member configured to be navigated through vasculature of a patient to a target treatment site and a plurality of therapeutic elements arranged around a distal portion of the elongated member, wherein each therapeutic element includes a needle including a first portion comprising a polymer and a second portion including a metal, wherein the second portion is configured to puncture a vessel wall at the target treatment site when the needle is extended radially relative to a longitudinal axis of the elongated member.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partially schematic illustration of a neuromodulation system configured in accordance with some examples of the present disclosure.

FIG. 2 illustrates a technique for accessing a renal artery and modulating renal nerves with the system of FIG. 1 in accordance with some examples of the present disclosure.

FIG. 3 is a conceptual illustration of an example sympathetic nervous system (SNS) illustrating how the brain communicated with the body via the SNS.

FIG. 4 is an enlarged anatomic view of nerves innervating a left kidney to form the renal plexus surrounding the left renal artery.

FIGS. 5 and 6 are anatomic and conceptual diagrams, respectively, of a human body depicting neural efferent and afferent communication between the brain and kidneys of the human body.

FIGS. 7 and 8 are anatomic views of the arterial vasculature and venous vasculature, respectively, of a human.

FIG. 9A is a conceptual diagram illustrating an example neuromodulation catheter including needles with a first portion including a polymer and second portion including a metal, in accordance with some examples of the present disclosure.

FIG. 9B is a conceptual diagram illustrating a schematic cross-sectional view of the neuromodulation catheter of FIG. 9A taken along line A-A with the example neuromodulation catheter in a deployed configuration, in accordance with some examples of the present disclosure.

FIG. 9C is a conceptual diagram illustrating schematic cross-sectional views of the neuromodulation catheter of FIG. 9B taken along line B-B with the example neuromodulation catheter in a deployed configuration, in accordance with some examples of the present disclosure.

FIG. 10A is a conceptual diagram illustrating an example neuromodulation catheter including polymer needles defining needle lumens and a wire disposed in each needle lumen, in accordance with some examples of the present disclosure.

FIG. 10B is a conceptual diagram illustrating schematic cross-sectional views of the neuromodulation catheter of FIG. 10A taken along line C-C with the example neuromodulation catheter in a deployed configuration, in accordance with some examples of the present disclosure.

FIG. 10C is a conceptual diagram illustrating schematic cross-sectional views of the neuromodulation catheter of FIG. 10B taken along line D-D with the example neuromodulation catheter in a deployed configuration, in accordance with some examples of the present disclosure.

FIG. 11 is a flow diagram illustrating an example method for performing neuromodulation of renal nerves using the neuromodulation catheter of FIGS. 9A-9C, in accordance with some examples of this disclosure.

### DETAILED DESCRIPTION

The present technology is directed to devices, systems, and techniques for neuromodulation, such as renal neuromodulation, using needles. Although renal neuromodulation is primarily described herein, devices, systems, and techniques described herein may be applied to other types of neuromodulation, such as neuromodulation performed on nerves other than the renal nerves, at sites other than within a renal vessel, or both. In general, the devices, systems, and techniques described herein may be used to perform neuromodulation from within any suitable anatomical lumen that has nerves adjacent to the anatomical lumen.

As used herein, the terms “distal” and “proximal” define a position or direction with respect to the treating clinician or clinician's control device (e.g., a handle assembly.) “Distal” or “distally” can refer to a position distant from or in a direction away from the clinician or clinician's control device. “Proximal” and “proximally” can refer to a position near or in a direction toward the clinician or clinician's control device.

Renal neuromodulation, such as renal denervation, may be accomplished using one or more of a variety of treatment modalities, including radio frequency (RF) energy, microwave, energy, ultrasound energy, a chemical agent, or the like. When using a chemical agent, a neuromodulation catheter may be delivered to a renal vessel, such as a renal artery, of a patient. The neuromodulation catheter may include at least one port or needle through which the chemical agent is delivered. The chemical agent may be selected to modulate activity of one or more renal nerves adjacent to the renal artery in which the neuromodulation is positioned. For example, the chemical agent may be a neurotoxic chemical selected to chemically ablate the one or more renal nerves near the renal artery.

The clinician may manipulate the distal portion of the neuromodulation catheter, for example, by rotating a proximal portion of the neuromodulation catheter about a longitudinal axis of the neuromodulation catheter, so that at least one therapeutic element is oriented towards the target treatment site. For instance, a neuromodulation catheter may include a distal portion configured to be positioned in a renal vessel of a patient, such as a main renal artery, an accessory renal artery, a branch vessel, or a renal vein. The distal portion may include a plurality of therapeutic elements arranged around an outer perimeter (e.g., referred to herein as a circumference, through the catheter may have other geometries in other examples) of the distal portion of the catheter. For example, the neuromodulation catheter may include three or more therapeutic elements arranged around a circumference of the distal portion of the catheter. Each therapeutic element may include a needle, and each needle may be configured to be radially extended from the neuromodulation catheter to pierce a wall of the renal vessel.

In accordance with examples of this disclosure, each needle may include a first portion and a second portion. The first portion may include a polymer and the second portion may include a metal. In some implementations, the first portion may be a proximal portion of the needle and the second portion may be a distal portion of the needle, such that the needle constitutes a metal-tipped polymer needle. The first portion of the needle may allow the needle to deflect in response to relative movement between the neuromodulation catheter and the vessel wall in which the needle(s) is deployed, while the metal portion of the needle may facilitate the needle(s) piercing the vessel wall.

In other implementations, the first portion may be a radially outer portion of the needle (e.g., an outer annular portion of the needle). The radially outer portion defines a needle lumen, in which a wire is disposed. The wire may extend to at or near a distal tip of the needle and facilitate the needle piercing the vessel wall. Once the needle is within the vessel wall at the desired position, the wire may be withdrawn proximally within the needle lumen until only the first portion of the needle is within the vessel wall. This may allow the needle to deflect in response to relative movement between the neuromodulation catheter and the vessel wall. A needle including both a first portion including a polymer and a second portion including a metal may allow the needle to deflect in response to catheter movement relative to the vessel wall.

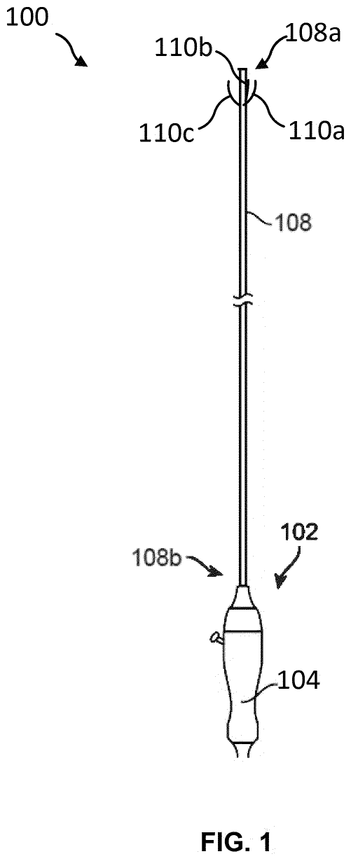

FIG. 1 is a partially schematic illustration of a neuromodulation system 100 (“system 100”) configured in accordance with some examples of the present technology. As shown in FIG. 1, system 100 includes a neuromodulation catheter 102. Neuromodulation catheter 102 includes a handle 104 and an elongated shaft 108 attached to the handle 104. The elongated shaft 108 may include a distal portion 108a and a proximal portion 108b. Distal portion 108a of neuromodulation catheter 102 include a plurality of therapeutic elements 110a-110c (“therapeutic elements 110”). Therapeutic elements 110 may be positioned around (e.g., distributed around) a perimeter of distal portion 108a, such as a circumference of distal portion 108a in examples in which distal portion 108a has a substantially circular cross-section.

Although distal portion 108a is shown in FIG. 1 as including three therapeutic elements 110 positioned around a circumference of distal portion 108a at a single longitudinal position along elongated shaft 108, in other examples, distal portion 108a may include any number of therapeutic elements 110. For example, distal portion 108a may include two, three, four, or more therapeutic elements 110 positioned around a circumference of distal portion 108a at a single longitudinal position. As another example, distal portion 108a may include positioned two, three, four, or more therapeutic elements 110 positioned around a circumference of distal portion 108a at each of multiple longitudinal positions along distal portion 108a. In examples in which distal portion 108a includes therapeutic elements 110 positioned at different longitudinal positions, each longitudinal position may include one or more therapeutic element 110, or one longitudinal position may include a different number of therapeutic elements 110 than one or more other longitudinal positions.

Elongated shaft 108 may have any suitable outer diameter, and the diameter can be constant along the length of elongated shaft 108 or may vary along the length of elongated shaft 108. In some examples, elongated shaft 108 can be 2, 3, 4, 5, 6, or 7 French or another suitable size.

Distal portion 108a of elongated shaft 108 is configured to be moved within a lumen of a human patient to locate therapeutic elements 110 at a treatment location site within or otherwise proximate to the lumen. For example, elongated shaft may be configured to position therapeutic elements 110 within a blood vessel, a ureter, a duct, an airway, or another naturally occurring lumen within the human body. In certain examples, intravascular delivery of the therapeutic elements 110 includes percutaneously inserting a guidewire (not shown) into a body lumen of patient and moving elongated shaft 108 and/or therapeutic elements 110 along the guidewire until therapeutic elements 110 reaches a target site (e.g., a renal artery). For example, the distal end of elongated shaft 108 may define a passageway for engaging the guidewire for delivery of therapeutic elements 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide catheter, sheath (not shown), or other guide device.

Once at the target site, therapeutic elements 110 can be configured to deliver therapy, such as RF energy, microwave energy, ultrasound energy, a chemical agent, or the like to provide or facilitate neuromodulation therapy at the target site. For ease of description, the following discussion will be primarily focused on delivering a chemical agent, such as a neurotoxic chemical, in which example therapeutic elements 110 include needles. It will be understood, however, that therapeutic elements 110 may include elements or structures configured to deliver other types of therapy. For example, therapeutic elements 110 may include needle electrodes configured to deliver RF energy for RF ablation of nerves near the lumen in which neuromodulation catheter 102 is positioned.

In examples in which neuromodulation catheter 102 is configured to deliver a chemical agent, therapeutic elements 110 may include needles configured to be deployed to extend radially from distal portion 108a and at least partially pierce a wall of the anatomical lumen in which distal portion 108a is positioned. The anatomical lumen may include a blood vessel, such as a renal artery, a renal vein, or the like. The following description primarily describes examples in which distal portion 108a is positioned in a renal vessel, although a person having ordinary skill in the art will appreciate that the techniques and devices described herein may be applied to other anatomical lumens. The needles may extend to and/or through the intima, media, and/or adventitia of the wall and be configured to deliver the chemical agent to the adventitia and/or periadventitial, in which renal nerves are located. By having therapeutic elements 110 located around a circumference of distal portion 108a, neuromodulation catheter 102 may be used to deliver the chemical agent around a circumference of the blood vessel. Although a circumference of the blood vessel is generally referred to herein, the blood vessel may not be perfectly circular in cross-section and may have any suitable geometry in cross-section.

In accordance with examples described herein, the needle of therapeutic element 110 (not pictured in FIG. 1) may include a first portion including a polymer and a second portion including a metal. The second portion may be configured to help the needle pierce or puncture a vessel wall at a target treatment site when the needle is extended radially relative to a longitudinal axis of elongated body 108. In some examples, the first portion may have sufficient stiffness along the longitudinal axis of therapeutic element 110 to facilitate the puncturing of a vessel wall with the needle. In some examples, the first portion may also be sufficiently flexible to deflect or bend upon the exertion of force along the longitudinal axis of elongated member 108 while the needle is deployed from the therapeutic element 110. E.g., due to physiological movement of a patient, inadvertent clinician manipulation, or the like.

In some implementations, the first portion may be a proximal portion of the needle and the second portion may be a distal portion of the needle, such that the needle constitutes a metal-tipped polymer needle. The second portion may be overmolded onto a proximal second of the first portion of the needle, may include a metal coating on at least a radially outer surface of a distal section of the polymer of the first portion. In either case, the second portion including the metal may include a biocompatible metal, such as stainless steel, titanium or a titanium alloy, or the like.

In other implementations, the first portion may be a radially outer portion of the needle (e.g., an outer annular portion of the needle). The radially outer portion defines a needle lumen, in which a wire is disposed. The wire may extend to at or near a distal tip of the needle and facilitate the needle piercing the vessel wall. Once the needle is within the vessel wall at the desired position, the wire may be withdrawn proximally within the needle lumen until only the first portion of the needle is within the vessel wall. This may allow the needle to deflect in response to relative movement between the neuromodulation catheter and the vessel wall. A needle including both a first portion including a polymer and a second portion including a metal may allow the needle to deflect in response to catheter movement relative to the vessel wall.

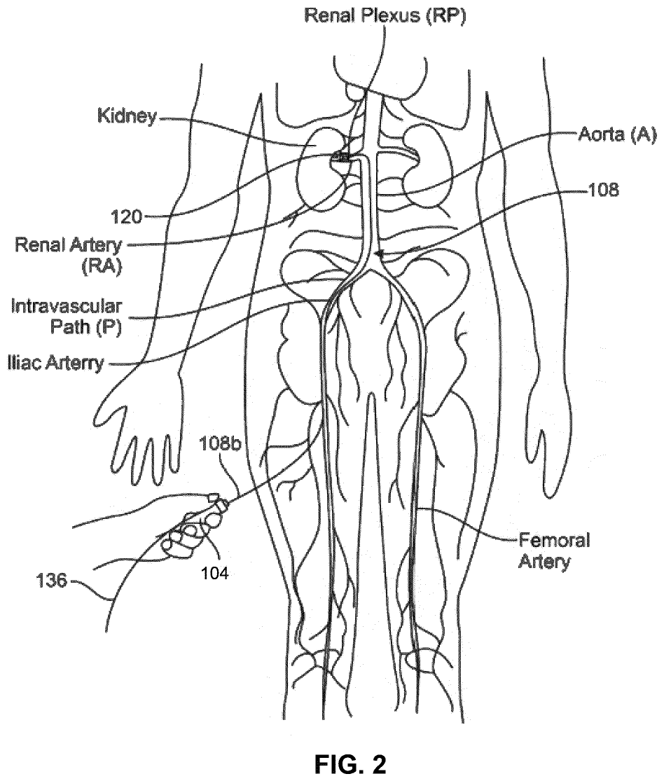

FIG. 2 (with additional reference to FIG. 1) illustrates an example technique for gaining access to renal nerves of an example patient in accordance with some examples of the present technology. Neuromodulation catheter 102 provides access to the renal plexus (RP) through an intravascular path (P), such as a percutaneous access site in the femoral (illustrated), brachial, radial, or axillary artery to a targeted treatment site within a respective renal artery (RA). By manipulating proximal portion 108b of elongated shaft 108 from outside the intravascular path (P), a clinician may advance at least distal portion 108a of elongated shaft 108 through the sometimes tortuous intravascular path (P) and remotely manipulate distal portion 108a (FIG. 1) of elongated shaft 108. Distal portion 108a may be remotely manipulated by the clinician using the handle 104.

In the example illustrated in FIG. 2, therapeutic elements 110 are delivered intravascularly to the treatment site using a guidewire 136 in an OTW technique. As noted previously, the neuromodulation assembly 120 may define a passageway for receiving guidewire 136 for delivery of the neuromodulation catheter using either an OTW or a RX technique. At the treatment site, guidewire 136 can be at least partially withdrawn or removed, and therapeutic elements 110 can transform or otherwise be moved to a deployed arrangement for delivering a chemical agent. In other examples, therapeutic elements 110 may be delivered to the treatment site within a different guide device, such as a guide sheath (not shown), with or without using guidewire 136. In examples in which the system includes a guide sheath, when therapeutic elements 110 are at the target site, the guide sheath may be at least partially withdrawn or retracted and therapeutic elements 110 may be transformed into the deployed arrangement. For example, at least a portion of therapeutic elements 110 may be self-expandable such that they expand to the deployed arrangement upon being released from the guide sheath. In still other examples, elongated shaft 108 may be steerable itself such that therapeutic elements 110 may be delivered to the treatment site without the aid of guidewire 136 and/or guide sheath.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for period of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally induced, chemically induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treatment tissue can include tissue at least proximate to a wall of the renal lumen. For example, with regard to a renal artery, a treatment procedure can include modulating nerves in the renal plexus, which lay intimately within or adjacent to the adventitia of the renal artery.

The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For examples, several properties of the renal vasculature may inform the design of treatment devices and associated methods for achieving renal neuromodulation via intravascular access and impose specific design requirements for such devices. Specific design requirements may include accessing the renal artery, positioning therapeutic elements 110 within the renal artery, delivering the chemical agent to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

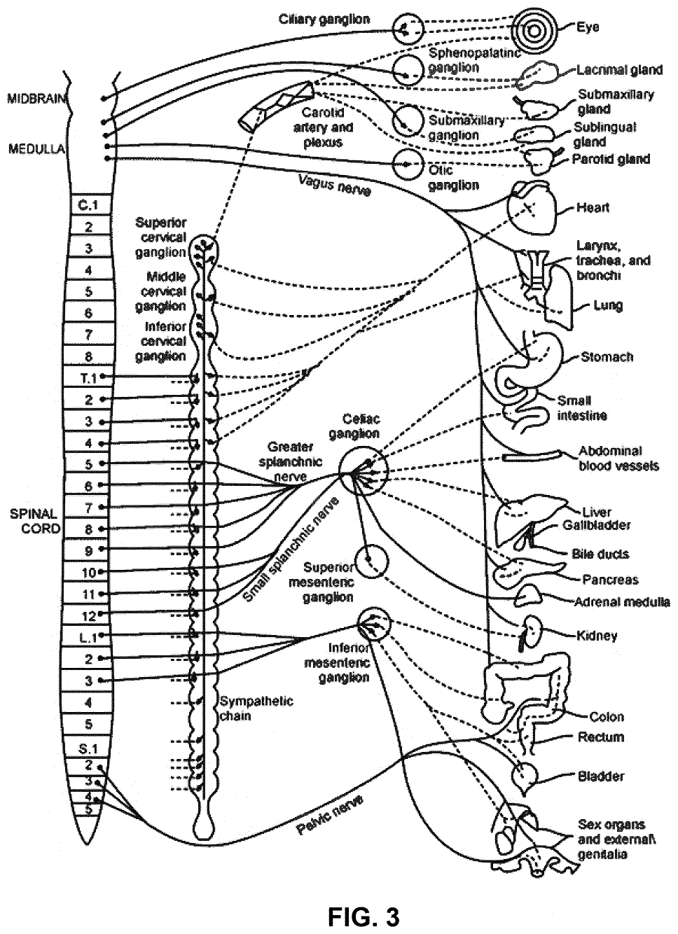

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are through to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia (which send sympathetic fibers to the gut).

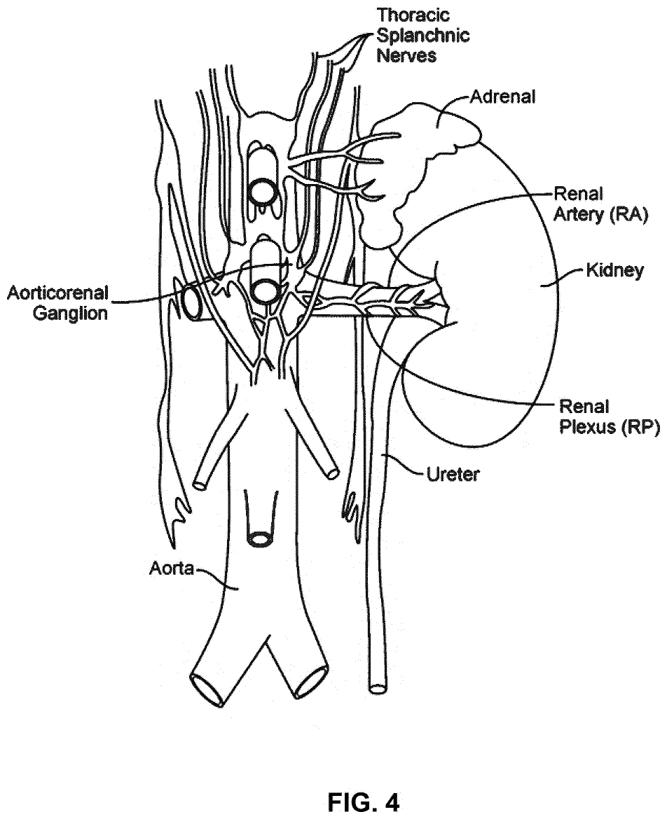

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise form the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

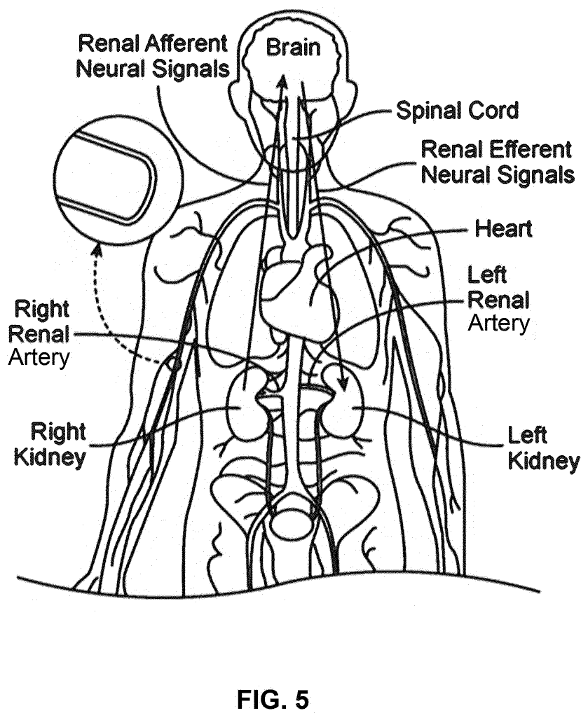

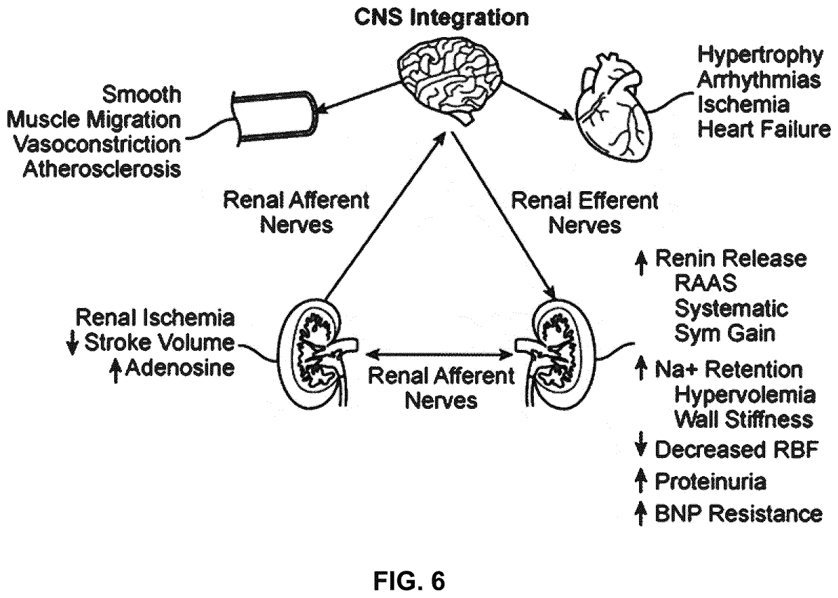

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome, and sudden death. Since the reduction of afferent neural signals contributing to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

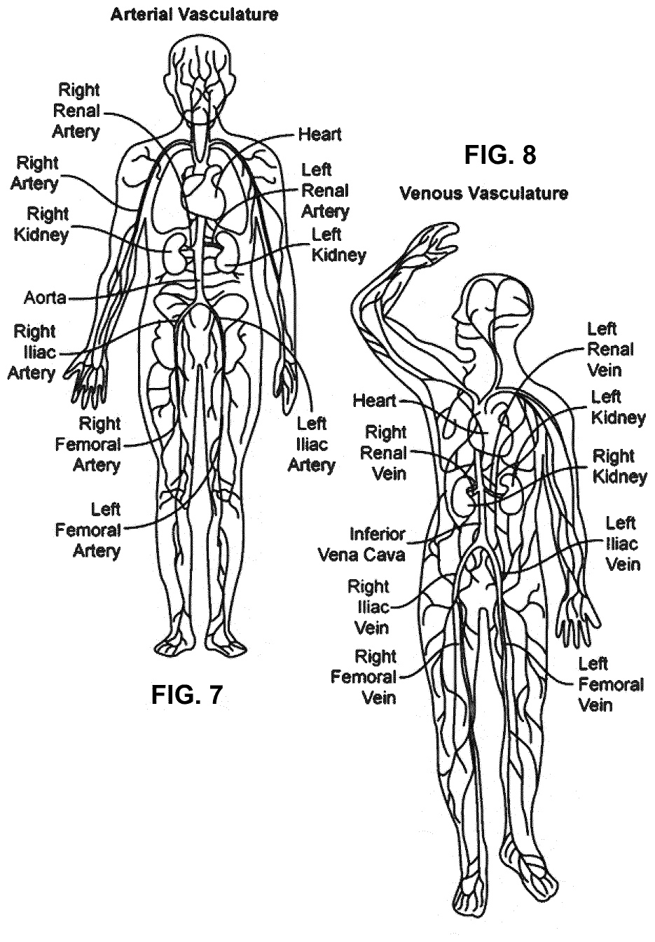

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of the therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that therapeutic elements 110 may intimately contact the vessel wall and/or extend at least partially through the vessel wall. Renal artery vessel diameter, DRA, typically is in a range of about 2-10 millimeters (mm), with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

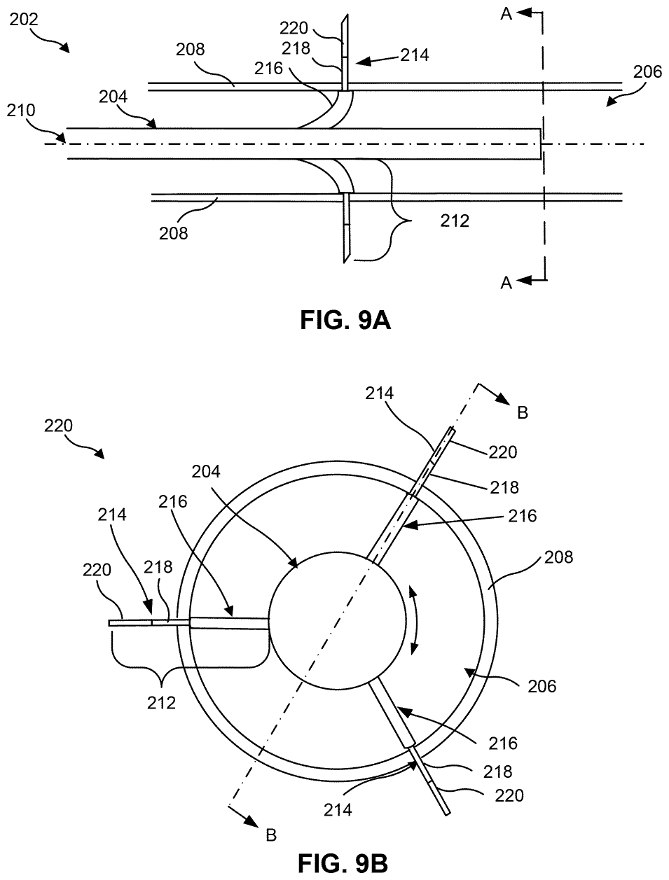

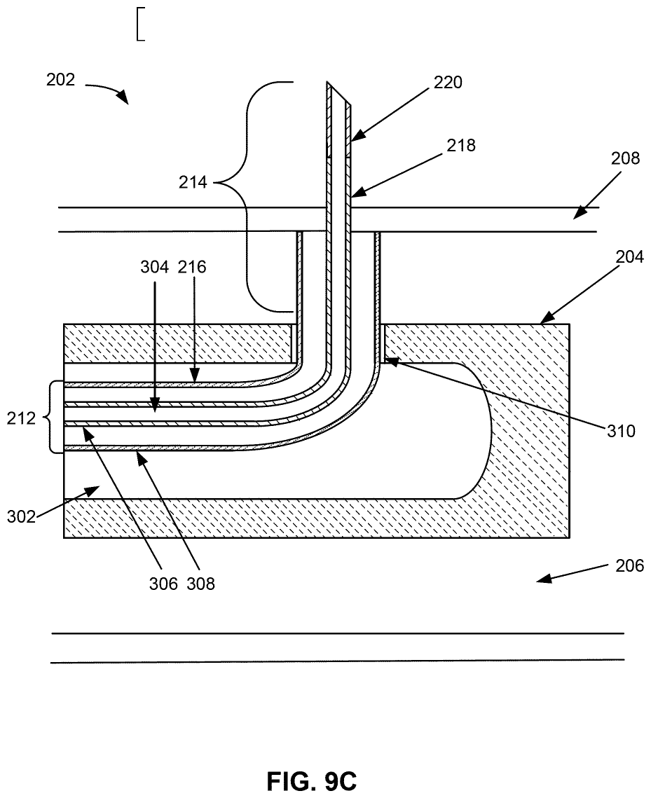

In accordance with examples of the current disclosure, a neuromodulation catheter may include therapeutic elements that include needles. The needles may be used for chemical neuromodulation, electrical signal delivery, e.g., for RF neuromodulation, or the like. In other words, the needles may include chemical delivery needles, needle electrodes, or needles that both allow chemical delivery and function as electrodes. FIG. 9A is a conceptual diagram of an example neuromodulation catheter 202 that includes an elongated shaft. FIG. 9A illustrates a distal portion 204 of the elongated shaft positioned within a renal artery 206. In other examples, distal portion 204 may be positioned within a different blood vessel, such as a renal vein, or another non-renal vessel. Neuromodulation catheter 202 is an example of neuromodulation catheter 102 of FIG. 1. The renal artery 206 includes vessel wall 208. In some instances, distal portion 204 may be positioned in a main renal artery, an accessory renal artery, a branch vessel extending distally from a main renal artery or accessory renal artery, or a renal vein. Neuromodulation catheter 202 includes a plurality of therapeutic elements 212 positioned about distal portion 204 (e.g., about a perimeter or circumference of distal portion 204).

Neuromodulation catheter 202 is configured to deliver a therapy via therapeutic elements. For example, neuromodulation catheter 202 may be configured to deliver a chemical agent, such as a neurotoxic chemical, through the plurality of therapeutic elements 212. As another example, neuromodulation catheter 202 may be configured to deliver an electrical therapy, such as RF ablation, via the plurality of therapeutic elements 212. The chemical agent may be selected to neuromodulate (e.g., chemically ablate) nerve tissue of the renal plexus adjacent to the renal artery 206 or facilitate other neuromodulation therapies (e.g., by affecting a conductivity of the tissue into which the chemical agent is injected). The chemical agent may include, for example, an alcohol, such as ethanol; distilled water; hypertonic saline; hypotonic saline; phenol; glycerol; lidocaine; bupivacaine; tetracaine; benzocaine; guanethidine; botulinum toxin; another neurotoxic fluid; or combinations thereof. In some examples, the chemical agent may be heated or cooled to additionally or alternatively thermally ablate nerve tissue of the renal plexus adjacent to the renal artery 206.

In the example shown in FIG. 9A, two therapeutic elements 212 are shown. However, neuromodulation catheter 202 includes three therapeutic elements 212, one of which is not shown because it is occluded by the body of neuromodulation catheter 202. In other examples, neuromodulation catheter 202 includes only one or two therapeutic elements 212 or more than three therapeutic elements 212. Each therapeutic element 212 includes a corresponding needle 214 and, in some, but not all, examples, a corresponding guide tube 216. Each guide tube 216 is configured to, for example, define a pathway through which a corresponding needle 214 may traverse to reach a target site for delivery of a therapy (e.g., a location of vessel wall 208). If an example therapeutic element 212 does not include a guide tube 216, then needle 214 may be configured to traverse a pathway to reach a target site for delivery of a therapy without a guide tube 216. Each therapeutic element 212 may have any suitable outer diameter and any suitable inner diameter, and the diameter can be constant along the length of elongated shaft 108 or may vary along the length of elongated shaft 108.

Therapeutic elements 212 are configured to be carried by neuromodulation catheter 202 in a delivery configuration as distal portion 204 of neuromodulation catheter 202 is advanced through vasculature of the patient to renal artery along a longitudinal axis 210. For example, in the delivery configuration, needles 214 may be withdrawn into guide tubes 216 and guide tubes 216 may be retracted within neuromodulation catheter 202. Therapeutic elements 212 are also configured to transform to a deployed configuration as shown in FIG. 9A. To transition to the deployed configuration from the delivery configuration, guide tubes 216 may be advanced out of neuromodulation catheter 202 to extend radially outwards relative to neuromodulation catheter 202 and contact the vessel wall 208 of renal artery 206, as shown in FIG. 9A. After guide tubes 216 have been advanced out of neuromodulation catheter 202 to contact the vessel wall 208, needles 214 may be advanced out of guide tubes 216 at least partially through vessel wall 208 (e.g., only partially through vessel wall 208 or completely through vessel wall 208). Alternatively, needles 214 may be fixed relative to guide tubes 216, such that guide tubes 216 and needles 214 are extended out of neuromodulation catheter 202 together and the exposed length of needles 214 out of guide tubes 216 is fixed. To transition back to the delivery configuration, guide tubes 216 and needles 214 may be retracted within neuromodulation catheter 202.

As another example, in the delivery configuration, needles 214 may be withdrawn into guide tubes 216 and guide tubes 216 may be urged against an outer surface of neuromodulation catheter 202 using a guide sheath (not shown in FIG. 9A). To transition to the deployed configuration, the guide sheath may be withdrawn proximally relative to neuromodulation catheter 202, which allows guide tubes 216 to expand radially outward. As such, in some examples, guide tubes 216 may be formed of a resilient material, such as a shape memory material, which is self-expanding upon being released from the guide sheath. Once guide tubes 216 have expanded to contact vessel wall 208, needles 214 may be advanced out of guide tubes 216 at least partially through vessel wall 208.

Needles 214 of neuromodulation catheter 202, as shown here in FIG. 9A, includes a first portion 218 and a second portion 220. In some examples, first portion 218 may form a proximal portion of needle 214 and second portion 220 may form a distal tip of needle 214.

First portion 218 may include a relatively flexible material, such as a polymer. The polymer may be sufficiently flexible to bend or deflect in response to relative movement between vessel wall 208 and distal portion 204 when needles 214 is deployed at least partially in vessel wall 208. This may reduce a risk of damage to vessel wall 208 by needles 214 in response to relative movement between neuromodulation catheter 202 and vessel wall 208 while one or more therapeutic elements 212 are in the deployed configuration. First portion 218 also may have sufficient column strength to transfer force applied by the clinician along needle 214 into second portion 220 when advancing needles 214 radially and allow needle 214 to pierce vessel wall 208.

In some implementations, first portion 218 also may include a reinforcement, such as a metal wire. For example, first portion 218 may include a shape memory wire embedded within first portion 218. The shape memory wire may improve pushability of first portion 218, urge first portion 218 toward a selected shape, or the like, while still allowing relatively high flexibility for first portion 218, e.g., compared to a metal needle.

For instance, first portion 218 may include a biocompatible polymer. The polymer may include, for example, a thermoplastic, such as an elastomer. In some examples, the elastomer may include a polyurethane, such as an aromatic thermoplastic polyurethane available under the trade designation Pellethane® available from The Lubrizol Corporation, Woickliffe, Ohio. In some examples, the elastomer may include copolymers, such as block copolymers comprising polyamide and polyether available under the trade name Pebax® available from Arkema S.A., Colombes, France. In some implementations, the polymer may have a Shore hardness of 80A or between 25D and 77D, such as between 55D and 77D. The polymer may be selected based at least in part on the dimensions of first portion 218, e.g., to balance a Shore hardness of the polymer and a thickness (e.g., wall thickness) of first portion 218.

Second portion 220 may include a biocompatible metal. Second portion 220 is configured to facilitate piercing of vessel wall 208. For instance, second portion 220 may include stainless steel, titanium, a nickel-titanium alloy, such as Nitinol®, or the like. Second portion 220 may extend from a distal tip of needles 214 to any suitable length along needles 214. In some examples, second portion 220 may extend a relatively short distance along needles 214, such that the relative stiffness of second portion 220 does not reduce flexibility of needles 214 along a significant length of needles 214. For instance, the length of second portion 220 may be such that, when needles 214 are deployed in vessel wall 208, second portion 220 is fully within vessel wall 208.

Needles 214 may be manufactured using any suitable technique. For instance, needles 214 may be made using an overmolding technique. In an overmolding technique, a second portion 220 may be placed in a mold and the polymer may be introduce into the mold to join to second portion 220 and form first portion 218. In some implementations, the polymer may be introduced into the mold using injection molding.

As another example, needles 214 may be formed by first forming first portion 218, e.g., using extrusion, a molding process, or the like. Second portion 220 then may be formed on first portion 220, e.g., using a vapor phase deposition process, such as physical vapor deposition, or the like.

FIG. 9B illustrates a schematic cross-sectional view of example neuromodulation catheter 202 of FIG. 9A taken along line A-A with the example neuromodulation catheter 202 in a deployed configuration. As shown in FIG. 9B, a clinician may manipulate distal portion 204 such that at least one of therapeutic elements 212 is oriented directly towards a treatment site (not pictured). In some examples, as shown in FIG. 9B, therapeutic elements 212 may be fully deployed such that the distal end of the guide tubes 216 contact the inner surface of vessel wall 208 and needles 214 extend into and/or through vessel wall 208. In some examples, as shown in FIG. 9B, second portions 220 may be fully within vessel wall 208, e.g., such that first portions 218 are present at the inner surface of vessel wall 208. In some examples, needles 214 may be extended prior to full deployment of therapeutic element 212. In other examples, needles 214 will be extended after the full deployment of therapeutic element 212 such that therapeutic elements 212 contact vessel wall 208. Extending needles 214 through vessel wall 208 may result in, upon delivery of a chemical agent, a chemical agent being directed out of needles 214 into tissue surrounding renal artery 206.

FIG. 9C illustrates a schematic cross-sectional view of example neuromodulation catheter 202 of FIG. 9B, taken along line B-B with the example neuromodulation catheter 202 in a deployed configuration. In the example shown in FIG. 9C, one therapeutic element 212 is shown. However, neuromodulation catheter 202 includes three therapeutic elements 212, two of which are not shown for simplicity of illustration. Therapeutic element 212 of neuromodulation catheter 202 includes a guide tube 216 as part of therapeutic element 212, but in other examples therapeutic element 212 may omit guide tube 216. Each therapeutic element 212 includes a guide tube 216, a needle 214 including a first portion 218 and a second portion 220, a needle actuating member 306, and a guide tube actuating member 308.

Neuromodulation catheter 202 may include a catheter lumen 302. Catheter lumen 302 may extend from a proximal portion of neuromodulation catheter 202 to distal portion 204. Catheter lumen 302 may have any suitable outer diameter, and the diameter can be constant along the length of elongated shaft 108 or may vary along the length of elongated shaft 108. Therapeutic elements 212 extend within catheter lumen 302. Therapeutic element 212 may be deployed from within the catheter lumen 302 out of exit port 310 when being extended into the deployed configuration. Therapeutic elements 212 also may be retracted from outside the distal portion 204 into catheter lumen 302 when transforming from the deployed configuration to the delivery configuration. A clinician may advance or retract the therapeutic element 212 relative to the distal portion 204 of neuromodulation catheter 202 by exerting either a pushing force or a pulling force, respectively, on guide tube actuating member 308, e.g., using an actuator at or near a proximal end of neuromodulation catheter 202.

In some needles 214, as pictured in FIG. 9C, first portion 218 forms a proximal base of needled 214 and second portion 220 forms a distal tip of needles 214.

Neuromodulation catheter 202 may include one or more actuating member configured to allow a physician to manipulate neuromodulation catheter 202 at or near a proximal end of neuromodulation catheter 202 to extend and retract therapeutic elements 212. For example, neuromodulation catheter 202 may include a needle actuating member 306 attached to needles 214 and configured to extend or retract the needles 214 relative to distal portion 204. Needle actuating member 306 may extend to at or near a proximal end of neuromodulation catheter 202, e.g., within catheter lumen 302. In some examples, neuromodulation catheter 202 may include a single needle actuating member 306 attached in common to all needles 214. In other examples, neuromodulation catheter 202 may include a needle actuating member 306 for each corresponding needle 214. In examples with a corresponding needle actuating member 306 for each needle, a clinician may extend or retract the needle by exerting a pushing or pulling force on the corresponding needle actuating member 306 via an actuator, such as a dial, thumbwheel, slide, or the like, respectively. Needle actuating member(s) 306 may include a push member, such as a metal or polymer hypotube, or the like. In some implementations, needle actuating member(s) 306 may be integral with and formed from the same material as first portion 218 of needle 214.

Similarly, neuromodulation catheter 202 includes at least one guide tube actuating member 308. Guide tube actuating member 308 may extend to at or near a proximal end of neuromodulation catheter 202, e.g., within catheter lumen 302. In some examples, neuromodulation catheter 202 may include a single guide tube actuating member 308 attached in common to all guide tubes 216. In other examples, neuromodulation catheter 202 may include a guide tube actuating member 308 for each corresponding guide tube 216. In examples with a corresponding guide tube actuating member 308 for each guide tube, a clinician may extend or retract the guide by exerting a pushing or pulling force on the corresponding guide tube actuating member 308 via an actuator, such as a dial, thumbwheel, slide, or the like, respectively. Guide tube actuating member(s) 308 may include a push member, such as a metal or polymer hypotube, or the like. In some implementations, guide tube actuating member(s) 308 may be integral with and formed from the same material as guide tubes 216.

In implementations in which needles 214 are fixedly attached to guide tubes 216, neuromodulation catheter 202 may include actuating member(s) that actuate both needles 214 and guide tube 216. Neuromodulation catheter 202 may include a single actuating member for all needles 214 and guide tube 216 or may include a corresponding actuating member for each needle/guide tube pair.

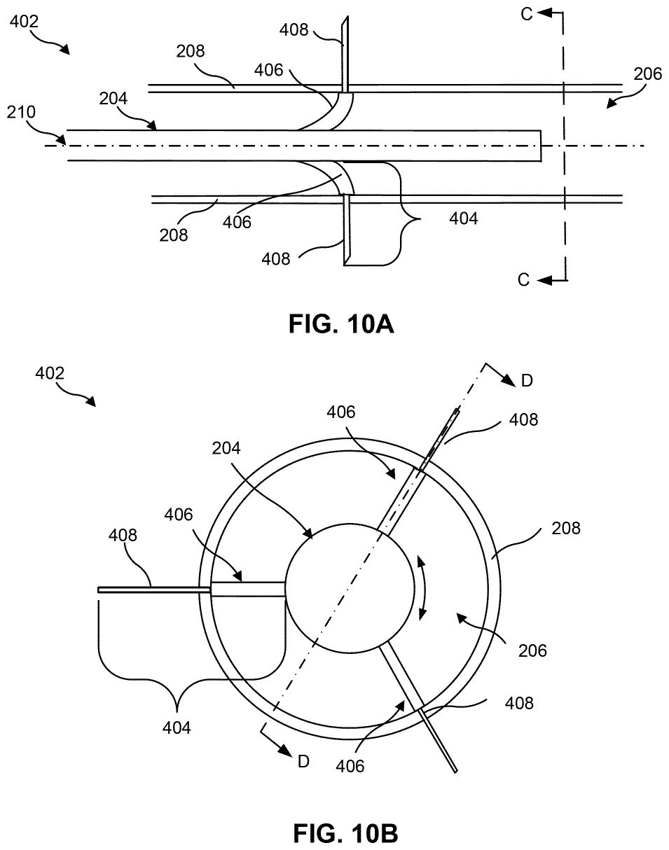

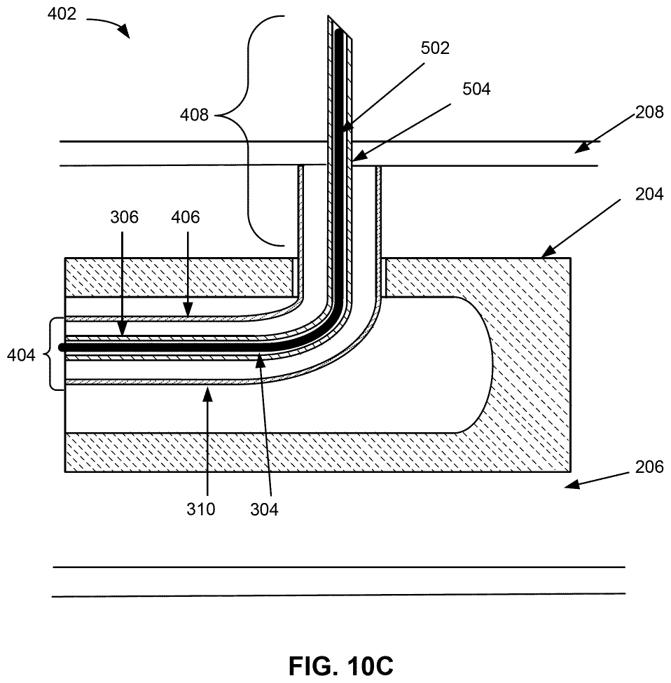

In some examples, rather than including a first portion 218 that is a proximal portion of needles 214 and a second portion 220 that is a distal portion of needles 214, the first and second portions may be concentric. For instance, FIG. 10A illustrates an example neuromodulation catheter 402 with a different needle configuration. Neuromodulation catheter 402 includes a plurality of therapeutic elements 404, each therapeutic element including a needle 408 and a guide tube 406 arranged around the distal portion 204 of an elongated body 108 (not pictured). FIG. 10B illustrates a schematic cross-sectional view of example neuromodulation catheter 402 of FIG. 10A taken along line C-C with the example neuromodulation catheter 402 in a deployed configuration. FIG. 10C illustrates a schematic cross-sectional view of example neuromodulation catheter 402 of FIG. 10A taken along line D-D with the example neuromodulation catheter 402 in the deployed configuration. The various components of the neuromodulation catheter 402 including, for example, the distal portion 204, the therapeutic elements 404, the guide tubes 406, and the like may be substantially similar to the components of neuromodulation catheter 202 described above, apart from any differences described below.

Needle 408 of neuromodulation catheter 402 includes a first, radially outer portion 504 including a polymer and a second, radially inner portion 502 including a metal (pictured in FIG. 10C). The materials for first portion 504 and second portion 502 may be selected from any of the materials described for first portion 218 and second portion 220, respectively. In some examples, first portion 504 may form the annular body of needle 408 and second portion 502 may form a relatively stiff core. In some implementations, with second portion 502 within the needle lumen, needle 408 may have sufficient column strength to pierce vessel wall 208 of renal artery 206 without substantial deflection or deformation in needle 408. In some examples, second portion 502 may provide needle 408 with sufficient column strength to pierce vessel wall 208 of renal artery 206 without needle 408 collapsing along the length of needle 408. When second portion 502 is withdrawn proximally into the elongated body of neuromodulation catheter 402, needle 408 may also have sufficient flexibility such that when a force is applied along the longitudinal axis 210 while the therapeutic element 404 is in the deployed configuration, needle 408 is able to deflect and pull the distal end of needle 408 away from vessel wall 208. This may reduce the risk of damage to vessel wall 208 by needle 408 if neuromodulation catheter 402 is moved along the longitudinal axis 210 while one or more therapeutic elements 404 are in the deployed configuration.

In some examples, a clinician may exert an actuating or pulling force on second portion 502 to extend or retract second portion 502 relative to first portion 504, respectively. In some examples, when withdrawn, second portion 502 may be positioned within needle lumen 306 within the elongated body of neuromodulation catheter 402. After needle 408 is extended and has penetrated vessel wall 208 of renal artery 206, a clinician may retract second portion 502, either partially or entirely, from the needle lumen 306 and deliver chemical agents into the tissue of the patient through the needle lumen 306. In some examples, after the clinician has finished delivering chemical agents, the clinician may then re-insert second portion 502 to help strengthen needle 408, e.g., in a delivery configuration in which first portion 504 is also withdrawn into the elongated body of neuromodulation catheter 402. The re-insertion of second portion 502 within needle 408 may be performed with first portion 504 in the deployed configuration or with first portion 504 retracted within distal portion 204 to neuromodulation catheter 402.

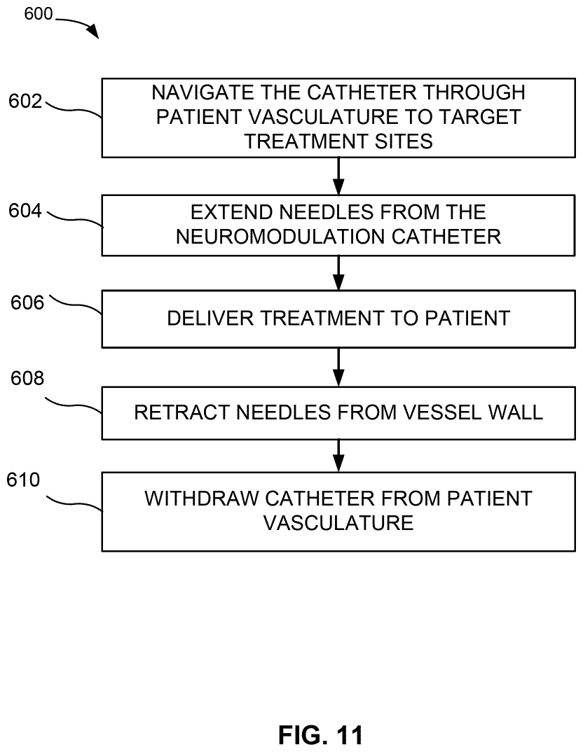

FIG. 11 illustrates an example method 600 of using an example neuromodulation catheter 202 of FIGS. 9A-C to deliver a treatment to tissue of a patient while positioned within renal artery 206. The technique includes navigating neuromodulation catheter 202 through patient vasculature to reach a target treatment site (602), extending one or more needles 214 from neuromodulation catheter 202 to pierce vessel walls 208 with the one or more needles 214 (604), delivering a neuromodulation treatment to the patient (606), retracting the one or more needles 214 from vessel wall 208 (608), and withdrawing neuromodulation catheter 202 from patient vasculature (610). While the method of FIG. 11 is directed to the neuromodulation catheter 202, it may also be used by clinicians for any other example neuromodulation catheter within this disclosure.

A clinician first navigates the neuromodulation catheter 202 through the vasculature of the patient to reach a target treatment site (602). In some examples, the clinician may navigate neuromodulation catheter 202 into renal artery 206 through a femoral artery approach, a brachial artery approach, a radial artery approach, or the like. The clinician may navigate the neuromodulation catheter 202 through patient vasculature using handle 104 of catheter 102, guidewire 136, or any other element configured to navigate a catheter through the patient vasculature (e.g., a guide sheath). The target treatment site may be any location where the clinician intends to deliver a neuromodulation treatment such as, but is not limited to, the renal nerve and tissues near vessel wall 208 of renal artery 206 or another suitable blood vessel. In some examples, a clinician may select a plurality of target treatment sites for treatment delivery.

After the clinician determines that neuromodulation catheter 202 is in the correct location (602), the clinician may extend one or more needles 214 from neuromodulation catheter 202 to pierce vessel walls 208 with the one or more needles 214 (604). In some examples, the clinician may first extend one or more guide tubes 216 to contact vessel wall 208 and approximately center distal portion 204 within the blood vessel. To move the one or more guide tubes 216 and/or needles, the clinician may interact with one or more actuator to actuate needle actuating member 306 and/or guide tube actuating member 308. In some examples, after extending one or more needles 214, the clinician may retract the one or more guide tubes 216.

In some examples, after the one or more needles 214 are extended into vessel wall 208, the clinician may deliver neuromodulation treatment to the patient (606) using the one or more needles 214 in accordance with the methods disclosed above. In other examples, the clinician may withdraw second portion 502 into needle lumen 304 prior to delivering neuromodulation treatment to the patient (606).

Upon the completion of delivering treatment to the patient (606), the clinician may retract the one or more needles 214 from vessel walls 208 and into the delivery configuration (608). In some examples the clinician may retract the one or more needles 214 using one or more needle actuating members 306. In some examples, if guide tubes 206 are in the extended configuration during neuromodulation treatment delivery (606), the clinician may further retract the one or more guide tubes 216 into the distal portion 204 of neuromodulation catheter 202 or adjacent to an outer surface of neuromodulation catheter 202. The clinician may retract the guide tubes 216 and therapeutic elements 212 using one or more guide tube actuating members 308. In another example, the clinician may retract the therapeutic elements 212 by retracting the distal portion 204 of the neuromodulation catheter 202 into a guide sheath (not picture in FIGS. 9A-9C) and forcing the therapeutic elements 212 into the guide sheath. Once the needles 214 are retracted into the therapeutic element 212, the clinician may withdraw neuromodulation catheter 202 from the patient vasculature (610).

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein. Additionally, while the description of the present technology is focused on delivering chemical agents, the present technology can equally be applied to other methods of neuromodulation therapy, including cooling, heating, electrical stimulation (using needle electrodes), RF energy delivery (using needle electrodes), or the like.

Certain aspects of the present disclosure described in the context of particular examples may be combined or eliminated in other embodiments. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single term exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additionally types of other features are not precluded.

Further disclosed herein is the subject-matter of the following clauses:
1. A catheter comprising:
   an elongated shaft configured to be navigated through vasculature of a patient to a target treatment site; and
   a plurality of therapeutic elements arranged around a distal portion of the elongated shaft, wherein each therapeutic element of the plurality of therapeutic elements comprises a needle comprising:
      a first portion comprising a polymer; and
      a second portion comprising a metal, the second portion being configured to puncture a vessel wall at the target treatment site when the needle is extended radially relative to a longitudinal axis of the elongated shaft.
2. The catheter of clause 1, wherein the first portion comprises a proximal portion of the needle.
3. The catheter of clause 1 or 2, wherein the second portion comprises a distal portion of the needle.
4. The catheter of clause 1, wherein the first portion comprises a circumferentially outer portion of the needle defining a needle lumen, and wherein the second portion comprises a wire disposed in the needle lumen.
5. The catheter of clause 4, wherein the second portion is configured to be retracted relative to the first portion while the first portion is substantially stationary relative to the elongated shaft.
6. The catheter of clause 5, wherein retraction of the second portion relative to the first portion leaves the needle lumen open and available to accept fluid flow therethrough.
7. The catheter of any one of clauses 1 to 6, wherein the first portion comprises a thermoplastic.
8. The catheter of clause 7, wherein the thermoplastic comprises a polyurethane.
9. The catheter of any one of clauses 1 to 8, wherein the second portion comprises a nickel-titanium alloy.
10. The catheter of any one of clauses 1 to 9, wherein each therapeutic element further comprises a guide tube, wherein the guide tube is configured to be extended from the elongated shaft to contact the vessel wall, and wherein the needle is configured to be extended from the guide tube to pierce the vessel wall.
11. The catheter of any one of clauses 1 to 10, wherein the first portion is configured to deflect in response to relative movement between the elongated shaft and the vessel wall.
12. The catheter of any one of clauses 1 to 11, further comprising a metal wire encapsulated by the first portion.
13. The catheter of any one of clauses 1 to 12, further comprising a plurality of actuating elements, wherein each actuating element of the plurality of actuating element is coupled to a corresponding needle of the plurality of needles, and wherein each actuating element is configured to transmit a force from adjacent the proximal end of the elongated shaft to the corresponding needle to cause the needle to move relative to the elongated shaft.
14. The catheter of clause 13, wherein the plurality of actuating elements comprises at least one of a pull member or a push member.
15. The catheter of clause 13 or 14, wherein the plurality of actuating elements extend to adjacent the proximal end of the elongated shaft.
16. The catheter of clause 13 or 14, wherein the plurality of actuating elements extend proximally from the corresponding needles to a common actuating element and attach to the common actuating element, and wherein the common actuating element extends to adjacent the proximal end of the elongated shaft.
17. A method comprising:
   navigating a catheter through vasculature of a patient to a target treatment site, wherein the catheter comprises:
      an elongated shaft; and
      a plurality of therapeutic elements arranged around a distal portion of the elongated shaft, wherein each therapeutic element of the plurality of therapeutic element comprises a needle comprising:
         a first portion comprising a polymer; and
         a second portion comprising a metal;
   extending at least one needle of the plurality of needles radially relative to a longitudinal axis of the elongated shaft; and
   puncturing a vessel wall at the target treatment site of the patient using the second portion of the at least one needle.
18. The method of clause 17, wherein the first portion comprises a proximal portion of the needles.
19. The method of clauses 17 or 18, wherein the second portion comprises a distal portion of the needles.
20. The method of clauses 17 or 18, wherein the first portion comprises a radially outer portion of the needle defining a needle lumen, and wherein the second portion comprises a wire disposed in the needle lumen.
21. The method of clause 20, further comprising retracting the second portion relative to the first portion while the first portion is substantially stationary relative to the elongated shaft.
22. The catheter of clause 21, wherein retracting of the second portion relative to the first portion leaves the needle lumen open and available to accept fluid flow therethrough.
23. The method of any one of the clauses 17 to 22, wherein the first portion comprises a thermoplastic.
24. The method of clause 23, wherein the thermoplastic comprises polyurethane.
25. The method of any one of clauses 17 to 24, wherein the second portion comprises a nickel-titanium alloy.
26. The method of any one of clauses 17 to 25, wherein each therapeutic element further comprises a guide tube, wherein the guide tube is configured to be extended from the elongated shaft to contact the vessel wall, and wherein the needle is configured to be extended from the guide tube to pierce the vessel wall.
27. The method of any one of clauses 17 to 26, wherein the first portion is configured to deflect in response to relative movement between the elongated shaft and the vessel wall.
28. The method of any one of clauses 17 to 27, wherein the catheter further comprises a metal wire encapsulated by the first portion.
29. The method of any one of clauses 17 to 28, wherein the catheter further comprises a plurality of actuating elements, wherein each actuating element of the plurality of actuating elements is coupled to a corresponding needle of the plurality of needles, and wherein each actuating element is configured to transmit a force from adjacent to the proximal end of the elongated shaft to the corresponding needle to cause the needle to move relative to the elongated shaft.
30. The catheter of clause 29, wherein the plurality of actuating elements comprises at least one of a pull member or a push member.
31. The catheter of clauses 29 or 30, wherein the plurality of actuating elements extend to adjacent the proximal end of the elongated shaft.
32. The catheter of clauses 30 or 31, wherein the plurality of actuating elements extend proximally from the corresponding needles to a common actuating element and attach to the common actuating element, and wherein the common actuating element extends to adjacent the proximal end of the elongated shaft.

1. A catheter comprising:
an elongated shaft configured to be navigated through vasculature of a patient to a target treatment site; and
a plurality of therapeutic elements arranged around a distal portion of the elongated shaft, wherein each therapeutic element of the plurality of therapeutic elements comprises a needle comprising:
a first portion comprising a polymer; and
a second portion comprising a metal, the second portion being configured to puncture a vessel wall at the target treatment site when the needle is extended radially relative to a longitudinal axis of the elongated shaft.

2. The catheter of claim 1, wherein the first portion comprises a proximal portion of the needle.

3. The catheter of claim 1, wherein the second portion comprises a distal portion of the needle.

4. The catheter of claim 1, wherein the first portion comprises a circumferentially outer portion of the needle defining a needle lumen, and wherein the second portion comprises a wire disposed in the needle lumen.

5. The catheter of claim 4, wherein the second portion is configured to be retracted relative to the first portion while the first portion is substantially stationary relative to the elongated shaft.

6. The catheter of claim 5, wherein retraction of the second portion relative to the first portion leaves the needle lumen open and available to accept fluid flow therethrough.

7. The catheter of claim 1, wherein the first portion comprises a thermoplastic.

8. The catheter of claim 7, wherein the thermoplastic comprises a polyurethane.

9. The catheter of claim 1, wherein the second portion comprises a nickel-titanium alloy.

10. The catheter of claim 1, wherein each therapeutic element further comprises a guide tube, wherein the guide tube is configured to be extended from the elongated shaft to contact the vessel wall, and wherein the needle is configured to be extended from the guide tube to pierce the vessel wall.

11. The catheter of claim 1, wherein the first portion is configured to deflect in response to relative movement between the elongated shaft and the vessel wall.

12. The catheter of claim 1, further comprising a metal wire encapsulated by the first portion.

13. The catheter of claim 1, further comprising a plurality of actuating elements, wherein each actuating element of the plurality of actuating elements is coupled to a corresponding needle, and wherein each actuating element of the plurality of actuating elements is configured to transmit a force from adjacent a proximal end of the elongated shaft to the corresponding needle to cause the needle to move relative to the elongated shaft.

14. The catheter of claim 13, wherein the plurality of actuating elements comprises at least one of a pull member or a push member.

15. The catheter of claim 13, wherein the plurality of actuating elements extend to adjacent the proximal end of the elongated shaft.

16. The catheter of claim 13, wherein the plurality of actuating elements extend proximally from the corresponding needles to a common actuating element and attach to the common actuating element, and wherein the common actuating element extends to adjacent the proximal end of the elongated shaft.

17. A method comprising:
navigating a catheter through vasculature of a patient to a target treatment site, wherein the catheter comprises:
an elongated shaft extending along a longitudinal axis; and
a plurality of therapeutic elements arranged around a distal portion of the elongated shaft, wherein each therapeutic element of the plurality of therapeutic element comprises a needle comprising:
a first portion comprising a polymer, and
a second portion comprising a metal;
extending at least one needle radially outwards relative to the longitudinal axis of the elongated shaft; and
puncturing a vessel wall at the target treatment site using the second portion of the at least one needle.

18. The method of claim 17, further comprising:
retracting the second portion relative to the first portion while the first portion is substantially stationary relative to the elongated shaft,
wherein retracting the second portion relative to the first portion facilitates fluid flow through the needle lumen.

19. A catheter comprising:
an elongated shaft extending along a longitudinal axis; and
a plurality of therapeutic elements arranged around a distal portion of the elongated shaft, each therapeutic element of the plurality of therapeutic elements comprising a needle comprising:
a radially outer portion defining a needle lumen, the outer portion comprising polymer; and
a wire disposed in the needle lumen, the wire comprising a metal, wherein the wire is configured to be advanced and retracted within the needle lumen.

20. The catheter of claim 19, wherein each therapeutic element further comprises a guide tube, wherein the guide tube is configured to be extended from the elongated shaft to contact a vessel wall, and wherein the needle is configured to be extended from the guide tube to pierce the vessel wall.
